(19) United States
(12) Patent Application Publication
(10) Pub. No.: US 2016/0061808 A1
(43) Pub. Date: Mar. 3, 2016

(54) MOLECULAR CELL IMAGING USING OPTICAL SPECTROSCOPY

(71) Applicant: Synaptive Medical (Barbados) Inc., Bridgetown (BB)

(72) Inventors: Aaron Cheung, Toronto (CA); Siu Wai Jacky Mak, Toronto (CA); Michael Wood, Toronto (CA); Cameron Piron, Toronto (CA)

(21) Appl. No.: 14/491,158

(22) Filed: Sep. 19, 2014

Related U.S. Application Data
(63) Continuation of application No. PCT/IB2014/064159, filed on Aug. 29, 2014.

(51) Int. Cl. G01N 33/483; A61B 10/02; G01N 21/27; G01N 21/65; G01N 21/47

(52) U.S. Cl. CPC G01N 33/4833; G01N 21/65; G01N 21/47; G01N 21/27; A61B 10/0266; G01N 2201/06113; G01N 2201/08

(57) ABSTRACT

The present invention provides a method and system for identification and isolation of cells from tissues using optical spectroscopy. The method includes accessing the cells using an access corridor, measuring the cells using optical spectroscopy, comparing the spectra of the cells to signature spectra, using the comparison to identify the cells and removing the cells into a container. The system includes an access corridor, a probe for measuring the cells, a resection tool, a collection tube and a collection container.

### FIELD

The present disclosure relates to imaging methods for use in minimally invasive therapy and image guided medical procedures using optical spectroscopy imaging of cells.

### BACKGROUND

Brain tumors are abnormal cell proliferations that occur in the central nervous system (CNS). It is estimated that there are over 23,000 new brain tumor cases in the United States (US) resulting in over 14,000 deaths per year (Ostrom et al., Neuro-oncology, 2013). Glioblastoma Multiforme (GBM), World Health Organization grade IV astrocytoma, is the most common and aggressive primary brain tumor in humans accounting for over 45% of all malignant brain tumors in the US (Ostrom et al., Neuro-oncology, 2013). The current standard care for GBM involves a combination of chemotherapy with the oral methylating agent, temozolomide, radiation therapy, and/or maximal surgical resection. Although tumor shrinkage is observed following such treatments, brain tumor relapse is often observed in around 90% of patients resulting in a median survival of only 12 to 15 months (Stupp et al., The lancet oncology, 2009; Weller et al., Neuro-oncology, 2013). Cancer stem cells (CSCs) may be behind the high occurrence of tumor relapse after initial cancer treatments.

There is evidence that cancer is maintained and driven by stem-like cells known as CSCs, similar to organs where maintenance and homeostasis are driven by adult stem cells (Beck and Blanpain, Nature reviews Cancer, 2013; Zhou et al., Nature reviews Drug discovery, 2009). CSCs were first isolated from leukemia (Bonnet and Dick, Nature medicine, 1997; Lapidot et al., Nature, 1994) and have since been isolated from many solid tumors including breast (Al-Hajj et al., Proceedings of the National Academy of Sciences of the United States of America, 2003; Ponti et al., Cancer research, 2005), colon (Dalerba et al., Proceedings of the National Academy of Sciences of the United States of America, 2007; O'Brien et al., Nature, 2007; Ricci-Vitiani et al., Nature, 2007; Vermeulen et al., Proceedings of the National Academy of Sciences of the United States of America, 2008), pancreatic (Hermann et al., Cell stem cell, 2007; Li et al., Cancer research, 2007), prostate (Collins et al., Cancer research, 2005; Patrawala et al., Oncogene, 2006), skin (Fang et al., Cancer research, 2005; Monzani et al., European journal of cancer, 2007; Quintana et al., Nature, 2008; Schatton et al., Nature, 2008), head and neck (Prince et al., Proceedings of the National Academy of Sciences of the United States of America, 2007), ovarian (Bapat et al., Cancer research, 2005; Curley et al., Stem cells, 2009; Szotek et al., Proceedings of the National Academy of Sciences of the United States of America, 2006; Zhang et al., Cancer research, 2008), lung (Eramo et al., Cell death and differentiation, 2008; Kim et al., Cell, 2005), and liver (Yang et al., Cancer cell, 2008). Brain tumor stem cells (BTSCs) were first isolated from post-operative brain tumor samples, including GBM, by sorting for surface markers that enriched for BTSCs in the CD133+ fraction (Singh et al., Cancer research, 2003; Singh et al., Nature, 2004). BTSCs exhibit properties of stem cells including their ability to self-renew in vitro as non-adherent neurospheres and multipotency in vitro, the ability to differentiate into the three neural lineages including neurons, astrocytes, and oligodendrocytes. They also exhibit the same properties in vivo where the injection of as few as 100 CD133+ cells intracranially into immunodeficient xenograft models are able to reinitiate brain tumors that phenocopy the original patient, demonstrating multipotency of the BTSCs in vivo but more importantly, the ability of BTSCs to reinitiate the brain tumor. Finally, BTSCs exhibit self-renewal properties in vivo as CD133+ BTSCs could be isolated from the brain tumors of primary xenografts and serially transplanted into secondary xenografts and reinitiate brain tumor formation. These results demonstrate that a small population of cells within the brain tumor exhibit stem cell properties which allow these cells to initiate brain tumor formation. It would therefore be of great advantage if cancer cells and in particular CSCs could be accurately and efficiently identified within normal tissue in vivo so they could be effectively targeted during therapy such as surgical resection. Note that the term CSCs may have different nomenclature in the field such as, but not limited to, tumor stem cells, tumor initiating cells, tumor progenitor cells, cancer initiating cells, or cancer progenitor cells. In this patent, the term CSCs encompasses all the cell types aforementioned. Similarly, this is also extended to BTSCs where the term brain tumor can be used as a prefix of the aforementioned terms to describe CSC within brain tumors.

The discovery of BTSCs is of huge significance because it may explain the high recurrence and mortality rates seen in brain tumor patients who have undergone standard care (Stupp et al., The lancet oncology, 2009; Weller et al., Neuro-oncology, 2013). One of the characteristics of CSCs is that they are able to evade many standard care treatments. For example, BTSCs have been shown to exhibit resistance to common antineoplastic chemotherapeutics drugs (Chen et al., Nature, 2012; Eramo et al., Cell death and differentiation, 2006) and to radiation therapy via preferential upregulation of DNA damage checkpoint response and increase in DNA repair capacity (Bao et al., Nature, 2006). This preferential chemo- and radiation-therapy resistance is not unique to CSCs of the brain but has also been shown for CSCs of breast (Diehn et al., Nature, 2009; Li et al., Journal of the National Cancer Institute, 2008), colon (Dylla et al., PloS one, 2008; Kreso et al., Science, 2013; Todaro et al., Cell stem cell, 2007), ovarian (Alvero et al., Cell cycle, 2009), pancreas (Adikrisna et al., Gastroenterology, 2012), and leukemia (Oravecz-Wilson et al., Cancer cell, 2009; Tehranchi et al., The New England journal of medicine, 2010). This demonstrates that therapeutic resistance is a common property of CSCs. By extension, surgical procedures may not be able to target the removal of CSCs of the tumor other than the bulk tumor itself. Therefore, the therapeutic resistance of BTSCs is a possible mechanism by which tumor relapse occurs as standard treatments are unable to target and remove BTSCs, leaving them behind in patients. The residual BTSCs are then able to reinitiate a tumor through their stem cell characteristics (self-renewal and multipotency) and cause recurrence. Consequently, it is important to be able to distinguish CSCs from other tumor cells because eradication of the CSCs may be required to eliminate the cancer. In this context, the term distinguish refers to the ability to create contrast or identify one cell type, such as CSCs, from another, such as non-CSCs, bulk tumor cells, adult stem cells, or healthy tissue.

Optical spectroscopy may be used to identify target cells such as CSCs. The optical absorption and scattering properties of biological tissue depend on both the chemical and structural properties of the tissue and the wavelength of the interacting light. How these absorption and scattering properties of tissue change as a function of light can be particularly useful, as it is often unique to chemicals or structures in the tissue (the spectrum of the tissue). For example the absorption features of oxy- and deoxy-hemoglobin can be used to measure the oxygenation of blood and tissue, and the scatter changes caused by different cellular sizes can be used to detect precancerous and cancerous tissue. The field of measuring these changes in optical properties as a function of light is known as spectroscopy and the device to measure the light at the various wavelengths is known as a spectrometer. Spectroscopy has found a wealth of current and potential applications in medicine.

An example of optical spectroscopy is Raman spectroscopy, a rapid and nondestructive method to analyze the chemistry of a given material using light (Raman and Krishnan, Nature, 1928). Raman spectroscopy takes advantage of an optical property known as inelastic scattering that occurs when light interacts with matter. This inelastic scattering is unique to the molecular structures of the matter, thus providing a unique spectra (or signature) of the matter that can be unambiguously distinguished and identified. Raman spectroscopy may provide neurosurgeons with an unambiguous and objective method to create contrast between tissues that are relevant to neurosurgery. For example, Raman spectroscopy may aid and assist neurosurgeons in distinguishing between healthy and tumor tissues, therefore minimizing the amount of tumour tissues left behind while preserving the critical healthy tissues, ultimately improving the surgical outcome of the patient. Studies using xenograft mouse models with transplanted brain tumor cells (Ji et al., Science translational medicine, 2013) and frozen human brain tumor sections (Kalkanis et al., Journal of neuro-oncology, 2014) have provided proof-of-principle of the potential of Raman spectroscopy in distinguishing healthy and tumor tissue. Raman measurements have been acquired from a number of different stem cell types ex vivo (Harkness et al., Stem cells and development, 2012; Hedegaard et al., Analytical chemistry, 2010) but have not yet been acquired from BTSCs or from CSCs in vivo.

Port-based surgery is a minimally invasive surgical technique where a port is introduced to access the surgical region of interest using surgical tools. Unlike other minimally invasive techniques, such as laparoscopic techniques, the port diameter is larger than tool diameter. Hence, the tissue region of interest is visible through the port.

Current methods of identifying healthy versus tumor tissue during port-based surgical procedures involve visual verification using an externally placed video scope. Visual verification is a subjective method. Tissue identification using a method such as optical spectroscopy would provide a quantitative means of effectively confirming tissue types during a surgical procedure.

Current standard care treatment from a surgical standpoint for brain tumor patients relies on the neurosurgeons' ability to distinguish tumour from healthy tissues based on preoperative images (i.e. Magnetic Resonance Imaging; MRI) and expertise (i.e. colour contrast between tissues), which is highly subjective. However, as noted above, even with maximal surgical resection, brain tumor recurrence remains the main cause of mortality for brain tumor patients post-treatment because neurosurgeons do not have the capability to directly visualize tumor tissue and BTSCs intraoperatively.

Thus, there is a need for optical spectroscopy as a tool for targeted cell identification in situ. As used herein, in situ means within the tissue of origin. There is also a need for rapid on-site diagnosis of resected tissues. There is a further need for direct assessment of resected tissue or cells with a reduction in steps between resection and assessment.

### SUMMARY

An object of the present invention is to provide systems, methods and devices for identifying target cells using optical spectroscopy in situ. A further object of the present invention is to provide systems, methods and devices for isolating cells from tissues using optical spectroscopy.

Thus by one broad aspect of the present invention, a method is provided for identifying cells comprising accessing the cells using an access corridor, measuring the cells using optical spectroscopy, comparing the spectra of the cells to a database of signature spectra or to spectra of adjacent cells, and using the comparison to identify the cells.

By another broad aspect of the present invention, a method is provided for isolating target cells from a tissue of a subject comprising accessing the cells using an access corridor, measuring the cells using optical spectroscopy via the access corridor, comparing the spectra of the cells to a database of signature spectra or to spectra of cells in surrounding tissue, using the comparison to determine the presence of target cells, and resecting the target cells into a container.

Another broad aspect of the present invention provides an interconnected system for the identification of cells comprising an access corridor for accessing the cells within a tissue, a probe connected to a means for measuring the cells by optical spectroscopy through the access corridor, a means for maintaining the probe in a sterile condition, a resection tool for providing suction and resection of tissue through the access corridor, a collection tube from the resection tool to a collection container, and a collection container for holding the resected tissue.

### BRIEF DESCRIPTION OF THE DRAWINGS

Embodiments will now be described, by way of example only, with reference to the drawings, in which:

FIG. 1 illustrates current methodologies used to surgically remove brain tumors, the isolation, characterization, and verification of BTSCs, and the opportunities BTSCs present.

FIG. 2 describes the role of CSCs in the context of brain tumors and their significance in brain tumor relapse.

FIG. 3 is a flow chart illustrating the processing steps involved in a port-based surgical procedure and the integration of an optical spectroscopic system, in this case, Raman spectroscopy, to identify and distinguish BTSCs from other tumor cells.

FIG. 4 illustrates the insertion of an access port into a human brain, for providing access to internal brain tissue during a medical procedure.

FIG. 5 illustrates the insertion of a catheter as an access port into the brain.

FIG. 6 illustrates the insertion of an access port into a human brain during a medical procedure.

FIG. 7 illustrates the use of a resection tool through the port.

FIG. 8 provides a schematic of a Raman system using transmissive grating.

FIG. 9 provides a schematic of a Raman system using reflective grating.

FIG. 10 illustrates a proposed alternate workflow to FIG. 1 where the limitations described by FIG. 1 have been addressed.

FIG. 11 illustrates use of a confocal Raman spectroscope to capture Raman spectra from stem cell populations.

FIG. 12 is a flow chart which describes how BTSCs may be harvested and characterized from brain tumor samples from patients.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure. The section headings used herein are for organizational purposes only and are not to be construed as limiting the subject matter described.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

As used herein, the term in situ means in the tissue of origin; the term in vivo means within a living organism; the term ex vivo means outside of a living organism; the term in vitro means within a culture dish, test tube or elsewhere outside a living organism; “target cells” means cells that are intended for identification or isolation; “non-target cells” means cells that are not intended for identification or isolation; “surrounding tissue” means tissue outside of the tissue being measured; “adjacent cells” means cells within the same tissue as the cells being measured; “Raman Spectroscopy” includes fiber-based Raman systems incorporating transmissive grating or reflective grating, other variations of Raman spectroscopy including but not limited to Coherent anti-stokes Raman Spectroscopy (CARS), Shifted-excitation Raman difference spectroscopy (SERDS) and stimulated Raman Spectroscopy (SRS) and non-fiber based Raman systems.

### Tissue Identification In Situ

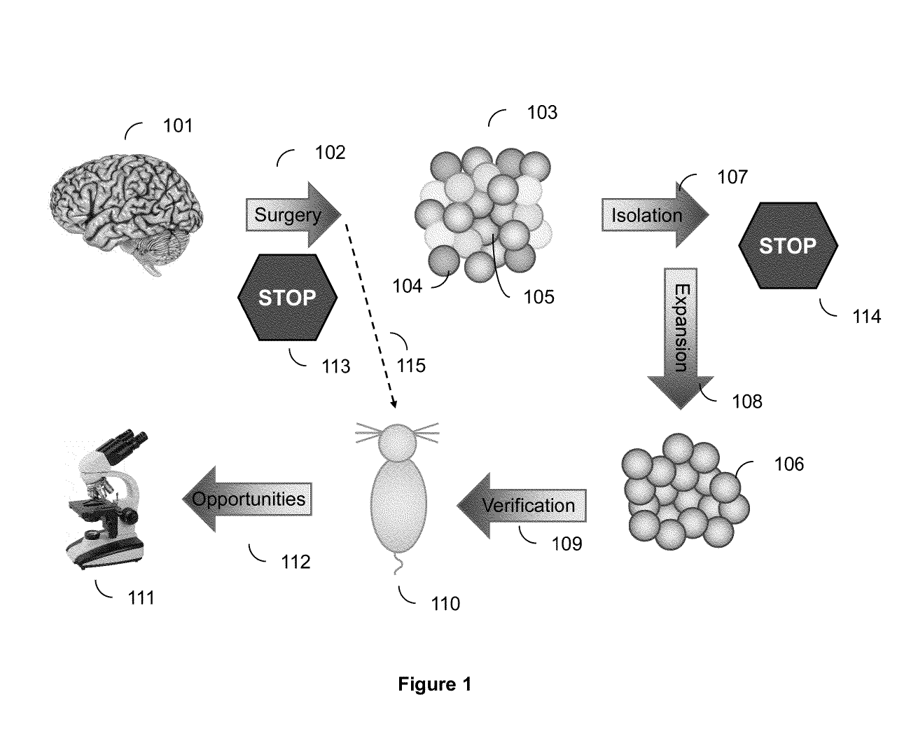

As an example of surgical removal of tumors, FIG. 1 illustrates the current methodologies used to surgically remove brain tumors, and isolate, characterize and verify target cells such as BTSCs. The workflow begins with a subject exhibiting a brain tumor 101 and to be treated using surgical resection 102 to remove the tumor. The subject may include a human. In brief, surgical removal of brain tumors involves the following steps: 1) Craniotomy—the temporary removal of a piece of bone in the skull to provide access to the dura; 2) Cut Dura—to provide temporary access to the brain; 3) Insertion of an access corridor such as a port and navigating it towards the target location, the brain tumor; 4) Resection—removal of the brain tumor using surgical tools; 5) Decannulation—removal of the access corridor after resection; and 6) closure and craniotomy.

Resection of brain tumor in the fourth step above is largely done in a non-targeted fashion. Available tools to neurosurgeons for removing brain tumors include using preoperative images such as MRI which becomes increasingly inaccurate intraoperatively as the brain shifts in position relative to the skull during surgery. Neurosurgeons also commonly use color contrast to distinguish between healthy and tumor tissue, which is highly subjective. Ultimately, the removal of brain tumor is largely performed using non-targeted and non-quantitative methods. For this reason, the extracted brain tumor 103 is also largely a heterogeneous population of cells that consists of both tumor mass cells 104 that make up the majority of the brain tumor and BTSCs 105 propagating the brain tumor. Note that it is likely that residual BTSCs are also left behind during neurosurgical removal of the brain tumor. Thus there are limitations to the current methods for distinguishing tumor from normal tissue intraoperatively.

### Cancer Stem Cells

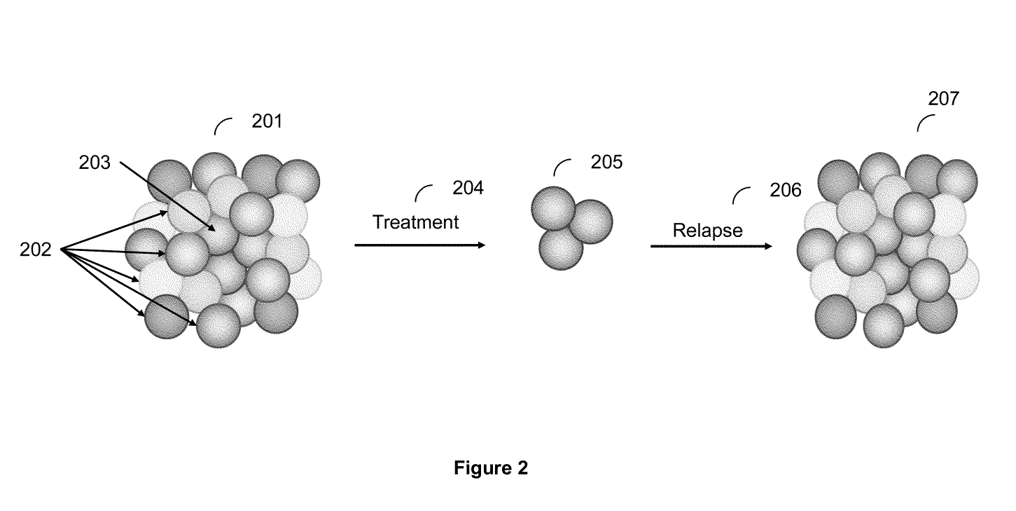

FIG. 2 illustrates CSCs in the context of a brain tumor. Note that the CSC model applies to other solid and hematologic tumors including, but not limited to, bone, breast, colon, head and neck, liver, lung, ovarian, pancreatic, prostate, and skin, and leukemia. FIG. 2 illustrates that a brain tumor 201 is comprised of a heterogeneity of cells illustrated by different shades. The majority of the cells 202 that make up the bulk of the brain tumor 201 include, but are not limited to, differentiated cells such as neurons, astrocytes, oligodendrocytes, and angiogenic cells. However, a minority of cells 203 within the brain tumor 201 are not differentiated and appear to be in a stem-like state referred to as BTSCs 203. BTSCs 203 exhibit the two cardinal properties of stem cells including their ability to self-renew and differentiate. BTSCs 203 tend to exist as a small percentage within the brain tumor but this is not a requirement.

Current standard care 204 for patients presenting with brain tumors involves a combination of chemotherapy with the oral methylating agent, temozolomide, radiation therapy, and/or maximal surgical resection. However, these treatments may be unable to target BTSCs as they exhibit chemotherapy and radiotherapy resistance. Furthermore, the inability for neurosurgeons to visualize the BTSCs prevents their targeted removal. The inability to target the BTSCs 203 directly for their removal results in the persistence of BTSCs 205 post-treatment. Post-treatment, although brain tumor patients are initially free of brain tumors, many patients experience relapse 206. There is evidence that residual BTSCs 205 that persist post-treatment are able to reinitiate tumor formation resulting in the recurrence of a brain tumor 207. Therefore, there is an urgent market need to remove BTSCs and more generally any type of CSC in a targeted manner.

Establishment of Brain Tumor Stem Cells from Resected Tissue

Returning to FIG. 1, current state of the art to establish CSC lines 106 from tumor samples 103 include multiple methods of isolation 107 and expansion 108 techniques. As an example, current methods for the isolation of BTSCs from brain tumors are provided here.

Isolation 107 of BTSCs 105 from a heterogeneous population of brain tumor 103 includes 1) the use of cell surface markers such as CD133 for sorting through flow cytometry and 2) the use of favorable conditions that promote self-renewal of normal neural stem cells such as the use of growth factors including Fibroblast Growth Factor 2 (FGF2) and Epidermal Growth Factor (EGF), extracellular matrix (ECM) including laminin and Poly-L Ornithine, and hypoxic oxygen concentrations such as 5% oxygen. The use of these techniques isolates BTSCs 105 from non-BTSCs 104 in the brain tumor sample 103.

Once BTSCs are isolated from the tumor cells, they are expanded. To expand 108 newly isolated BTSCs 107, BTSCs can be propagated in vitro by several methods including 1) non-adherent or 2) adherent methods. In the non-adherent method, BTSCs are typically propagated in a low attachment container in the favorable conditions described above (growth factors and oxygen concentrations) promoting BTSCs to adhere to each other rather than the container and form spheres of cells known as neurospheres. These neurospheres can then propagate and expand in this configuration. In the adherent method, BTSCs are typically propagated in a container coated with a favorable ECM, promoting their attachment to the container. BTSCs will then be propagated in favorable conditions described above (growth factors and oxygen tension). These BTSCs, described as monolayers, can then propagate and expand in this configuration. Once BTSCs are stably propagating in vitro, they are considered a BTSC line 106.

Prior to the use of BTSCs for research purposes, it is important to verify 109 their stem cell properties and confirm their identity as BTSCs. Similar to other stem cells, BTSCs should possess the two properties of stem cells, self-renewal and multipotency. Both these properties can be demonstrated in vitro where self-renewal is demonstrated via the routine propagation of the BTSCs as neurospheres or as a monolayer described above. Multipotency, or the ability to differentiate, can be demonstrated in vitro by placing BTSCs in differentiation inducing conditions by removing them from the self-renewal conditions described above (growth factors, oxygen tension, and substrate). For example, BTSCs can be placed in media lacking self-renewing factors FGF2 and EGF to promote their differentiation into the three neural lineages, neurons, astrocytes, and oligodendrocytes, which can then be confirmed by molecular techniques including, but not limited to, immunocytochemistry and quantitative polymerase chain reaction. It is also vital to verify the stem cell properties of BTSCs in an animal or in vivo, where in vivo means within a living organism. Typically, to perform in vivo characterization, a xenograft is done, that is, BTSCs are injected intracranially into another species 110. The xenograft host is usually an immunocompromised mouse. Multipotency is demonstrated by the development of a brain tumor in the xenograft host by the injected BTSCs 106. The brain tumor in the xenograft should reflect pathologically the original brain tumor 101 in the subject, demonstrating the BTSCs' ability to differentiate into the different cell types comprising the original brain tumor. To demonstrate self-renewal in vivo, serial transplantation can be performed. This is demonstrated by isolating BTSCs from the brain tumor formed in the xenograft 110, re-transplanting into a secondary xenograft recipient, and showing that a brain tumor can form again in the secondary xenograft. In theory, this can be performed over multiple serial transplantations demonstrating the self-renewal of BTSCs in vivo.

Upon verifying that BTSC lines have these stem cell properties, they are suitable for future use, including research 111, experimentation, and other opportunities 112. Altogether, FIG. 1 describes the current state of the art and common methodologies for the isolation of BTSCs 105 from a brain tumor 101 sample surgically removed from a subject and establishing a BTSC line 106.

There are two main limitations to the current methods used for BTSC isolation described above. The first limitation 113 is that there are currently no methods to remove BTSCs in a targeted manner when performing surgical resection in situ during brain tumor removal. For this reason, the removed brain tumor 103 is largely heterogeneous and may contain BTSCs 105, but most likely some BTSCs are also left behind within the patient which could explain the high recurrence and relapse rates of brain tumor patients. The second limitation 114 is the current need to culture BTSCs in vitro during the isolation 107 and expansion 108 phase. This is problematic as the culturing of BTSCs 106 in vitro can impose artifacts, such as, but not limited to, genetic and epigenetic changes, such that the BTSCs 106 do not resemble their in vivo state. The current norm of studying BTSCs that have been cultured in vitro may impact and confound any opportunities 112 such as research 111 to be performed on such BTSCs, yielding data that may not be relevant to their in vivo counterparts. There is currently a need to culture BTSCs in vitro because there are no methods to directly isolate the BTSCs in situ during surgery 102.

### EXAMPLE 1

### Method for In Situ Identification of Target Cells

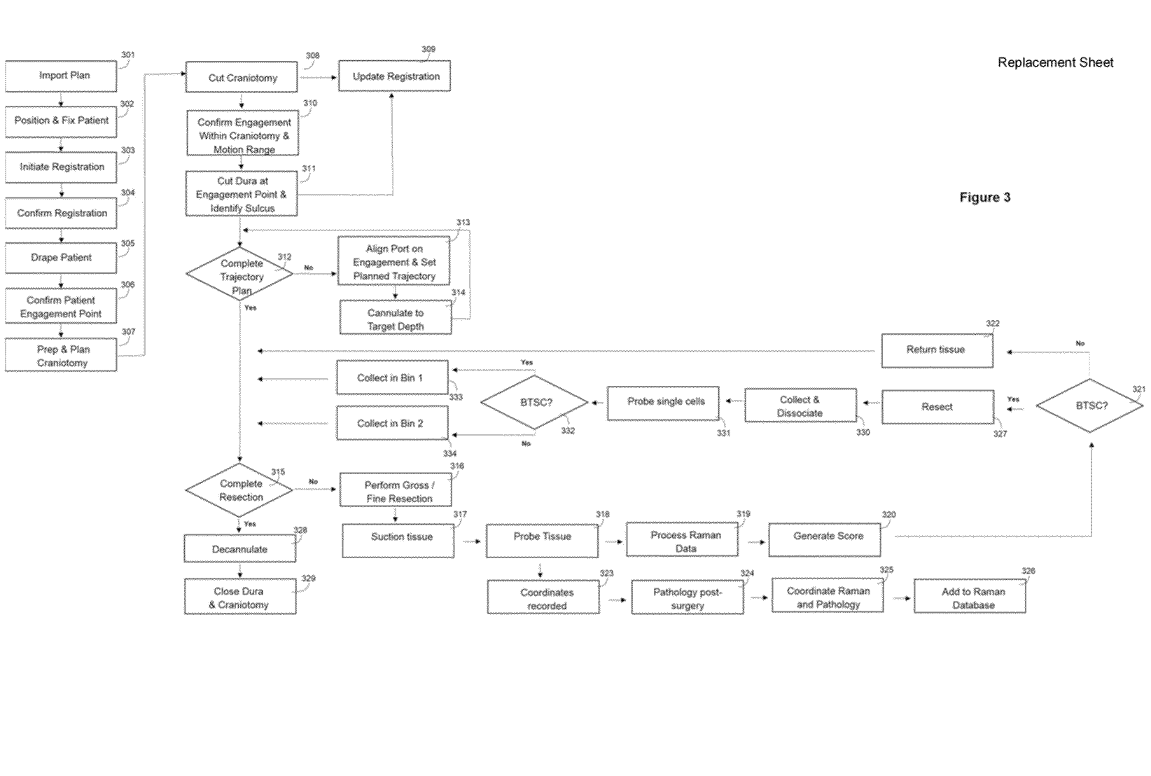

To overcome the first problem of identifying target cells in situ, a method is described here for optical spectroscopy in a subject. FIG. 3 is a flow chart illustrating the processing steps involved in a port-based surgical procedure using a navigation system. The example here describes identification of BTSCs but those skilled in the art will recognize that the method can be applied to other target cells, such as but not limited to other CSCs.

### A. Surgical Preparation

Surgical procedures are well known in the art. A first step involves importing a port-based surgical plan 301. An exemplary plan may include preoperative 3D imaging data (i.e., MRI, ultrasound, etc.), overlaying received inputs (i.e., sulci entry points, target locations, surgical outcome criteria, additional 3D image data information) on the preoperative 3D imaging data and displaying one or more trajectory paths based on the calculated score for a projected surgical path. An example of a process to create and select a surgical plan is outlined in the disclosure “PLANNING, NAVIGATION AND SIMULATION SYSTEMS AND METHODS FOR MINIMALLY INVASIVE THERAPY”, International Patent Application CA2014050272 which claims priority to United States Provisional Patent Application Ser. Nos. 61/800,155 and 61/924,993, which are hereby incorporated by reference in their entirety. The aforementioned surgical plan may be one example; other surgical plans and/or methods may also be envisioned.

Once the plan has been imported into the navigation system 301, the subject is affixed into position using a head or body holding mechanism. The head position is also confirmed with the subject plan using the navigation software 302.

The next step is to initiate registration of the subject 303. The phrase “registration” or “image registration” refers to the process of transforming different sets of data into one coordinate system. Data may be multiple photographs, data from different sensors, times, depths, or viewpoints. The process of “registration” is used in the present application for medical imaging in which images from different imaging modalities are co-registered. Registration is necessary in order to be able to compare or integrate the data obtained from these different modalities.

Those skilled in the art will appreciate that there are numerous registration techniques available and one or more of them may be used in the present application. Non-limiting examples include intensity-based methods which compare intensity patterns in images via correlation metrics, while feature-based methods find correspondence between image features such as points, lines, and contours. Image registration algorithms may also be classified according to the transformation models they use to relate the target image space to the reference image space. Another classification can be made between single-modality and multi-modality methods. Single-modality methods typically register images in the same modality acquired by the same scanner/sensor type, for example, a series of MR images can be co-registered, while multi-modality registration methods are used to register images acquired by different scanner/sensor types, for example in MRI and PET. In the present disclosure multi-modality registration methods are used in medical imaging of the head/brain as images of a subject are frequently obtained from different scanners. Examples include registration of brain CT/MRI images or PET/CT images for tumor localization, registration of contrast-enhanced CT images against non-contrast-enhanced CT images, and registration of ultrasound and CT.

Once registration is confirmed 304, the subject is draped 305. Typically draping involves covering the subject and surrounding areas with a sterile barrier to create and maintain a sterile field during the surgical procedure. The purpose of draping is to eliminate the passage of microorganisms (i.e., bacteria) between non-sterile and sterile areas.

Upon completion of draping 305, the next step is to confirm subject engagement points 306 and then prepare and plan craniotomy 307.

Upon completion of the preparation and planning of the craniotomy step 306, the craniotomy is carried out 308 in which a bone flap is temporarily removed from the skull to access the brain. Registration data is updated with the navigation system at this point 309.

The next step is to confirm the engagement within the craniotomy and the motion range 310. Once this data is confirmed, the procedure advances to the next step of cutting the dura at the engagement points and identifying the sulcus 311. Registration data is also updated with the navigation system at this point 309.

In an embodiment, by focusing the camera's gaze on the surgical area of interest, this registration update can be manipulated to ensure the best match for that region, while ignoring any non-uniform tissue deformation affecting areas outside of the surgical field. Additionally, by matching overlay representations of tissue with an actual view of the tissue of interest, the particular tissue representation can be matched to the video image to ensure registration of the tissue of interest. For example, the embodiment can:
Match video of post craniotomy brain (i.e. brain exposed) with imaged sulcal map;
Match video position of exposed vessels with image segmentation of vessels;
Match video position of lesion or tumor with image segmentation of tumor; and/or
Match video image from endoscopy up nasal cavity with bone rendering of bone surface on nasal cavity for endonasal alignment.

In other embodiments, multiple cameras may be used and overlaid with tracked instrument(s) views, and thus allow multiple views of the data and overlays to be presented at the same time, which may provide even greater confidence in a registration, or correction in more dimensions/views than provided by a single camera.

Thereafter, the cannulation process is initiated 312. Cannulation involves inserting a port into the brain, typically along a sulci path as identified in step 311, along a trajectory plan. Cannulation is an iterative process that involves repeating the steps of aligning the port on engagement and setting the planned trajectory 313 and then cannulating to the target depth 314 until the complete trajectory plan is executed 312.

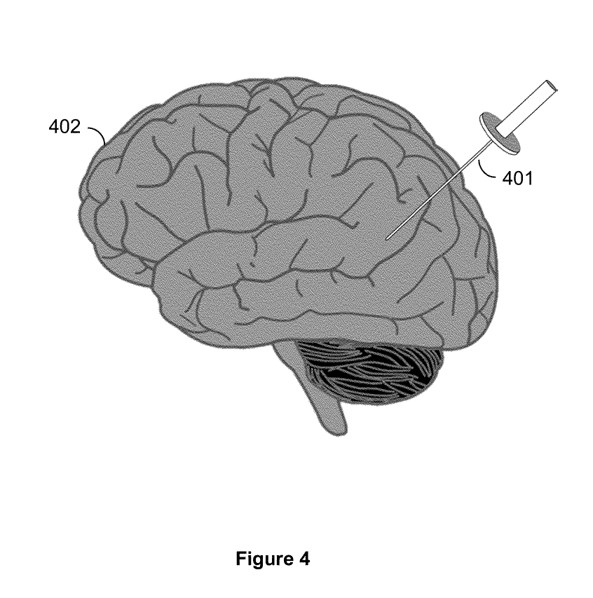

As an example of cannulation, FIG. 4 illustrates the insertion of an access port into a human brain, for providing access to internal brain tissue during a medical procedure. In FIG. 4, access port 401 is inserted into a brain 402, providing access to internal brain tissue. Access port 401 may include such instruments as catheters, surgical probe or cylindrical ports such as the NICO BrainPath. Surgical tools and instruments may then be inserted within the lumen of the access port in order to perform surgical, diagnostic or therapeutic procedures, such as resecting tumors as necessary.

During port-based surgery, a straight (linear) access port 401 is typically guided down a sulci path of the brain. Surgical instruments would then be inserted down the access port 401.

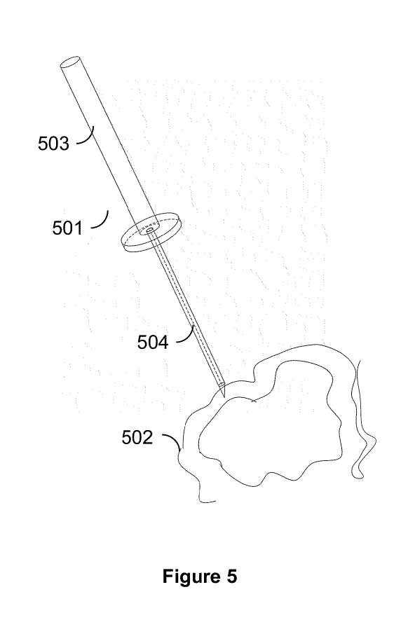

As another example, FIG. 5 illustrates the insertion of a catheter as an access port into the brain. In FIG. 5, catheter 501 is an access port positioned to navigate a brain 502. Catheter 501 is composed of a handle 503 at the proximal end and a linear (straight) probe 504 at the distal end. Probe 504 may be a resection tool, an image sensor and/or other types of sensing tools that can take measurements in different imaging modalities (e.g. ultrasound, Raman, OCT, PET, MRI, etc.).

As a further example, a new approach to resection of brain tumors is the use of a small port to access the tumor. The port is typically a hollow tube inserted into the brain for the purpose of minimally-invasive neurosurgery. The port is inserted via a burr hole craniotomy into a brain. Resection of the tumor is conducted via instruments inserted into the port.

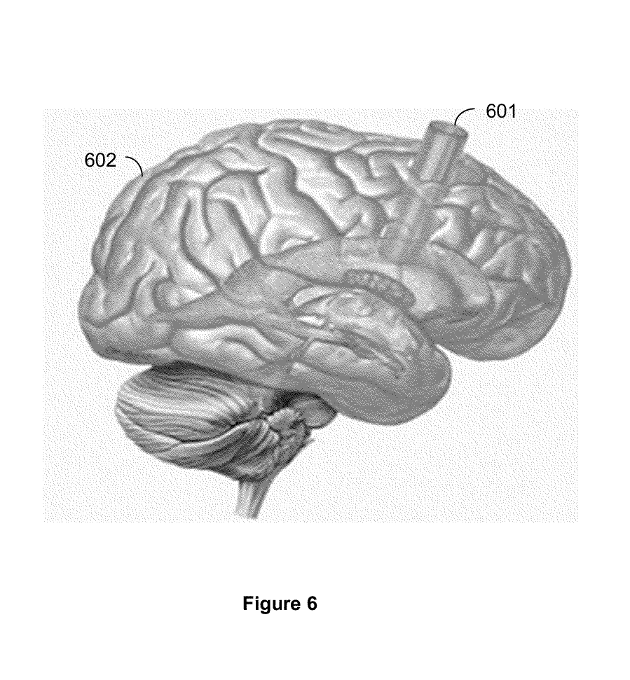

FIG. 6 shows an access port 601 inserted into a human brain 602, providing access to internal brain tissue. Surgical tools and instruments may then be inserted within the lumen of the access port in order to perform surgical, diagnostic or therapeutic procedures, such as resecting tumors as necessary. This approach allows a surgeon, or robotic surgical system, to perform a surgical procedure involving tumor resection in which the residual tumor remaining after is minimized, while also minimizing the trauma to the intact white and grey matter of the brain.

Returning to FIG. 3, the surgeon then performs resection 315 to remove part of the brain and/or tumor of interest. Resection 315 is a continual loop including both fine and gross resection 316.

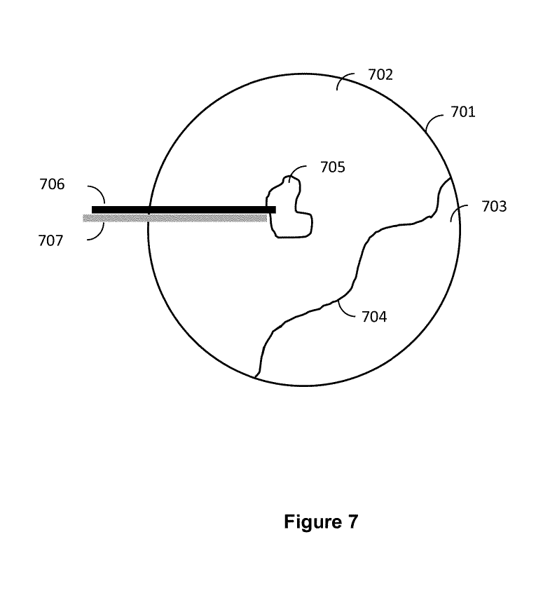

During resection, the surgeon makes use of a resection tool within the port as described above and as further illustrated in FIG. 7. The port 701 is inserted during the cannulation process which provides the surgeon with a view of the tissue lying beneath. This tissue could represent both the tumor area 702 and/or the healthy area 703 separated by a tumor boundary 704. A portion of the tissue beneath the port may also represent the location of the BTSCs 705 which the surgeon does not know a priori. During resection, a surgeon typically will use a resector tool 706 that has two functions, the first of which is to suction the tissue within the tool, and the second of which is to resect the tissue within the tool.

### B. In Situ Diagnostic Imaging

The resection tool is combined with means for imaging modalities. As a non-limiting example the resection tool is combined with a Raman probe 707. The Raman probe houses the fiber bundle which excites the tissue captured by the resection tool with a laser and detects the refracted light. The Raman probe or other imaging tools may be sterilizable or may be fitted with a sterile sleeve or other means to provide a sterile outer surface for the probe. The sterile sleeve may also allow the probe to be liquid-resistant, thereby allowing the probe to go through fluid/liquid within the surgical field and provide direct contact with tissue. The Raman probe is connected to a spectroscope at its distal end to analyze the Raman shift.

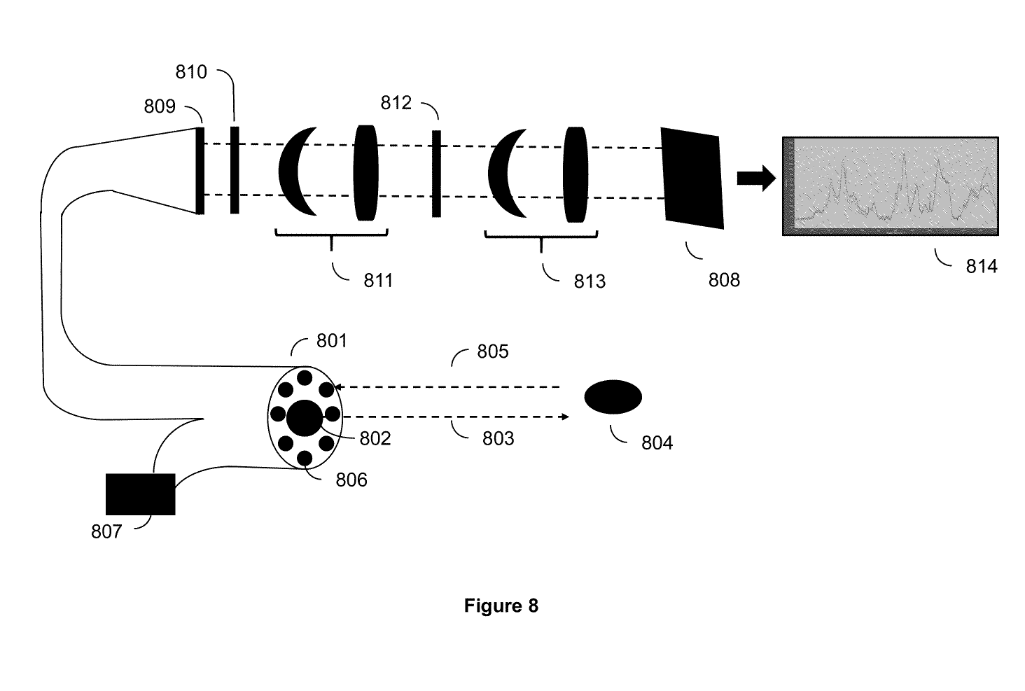

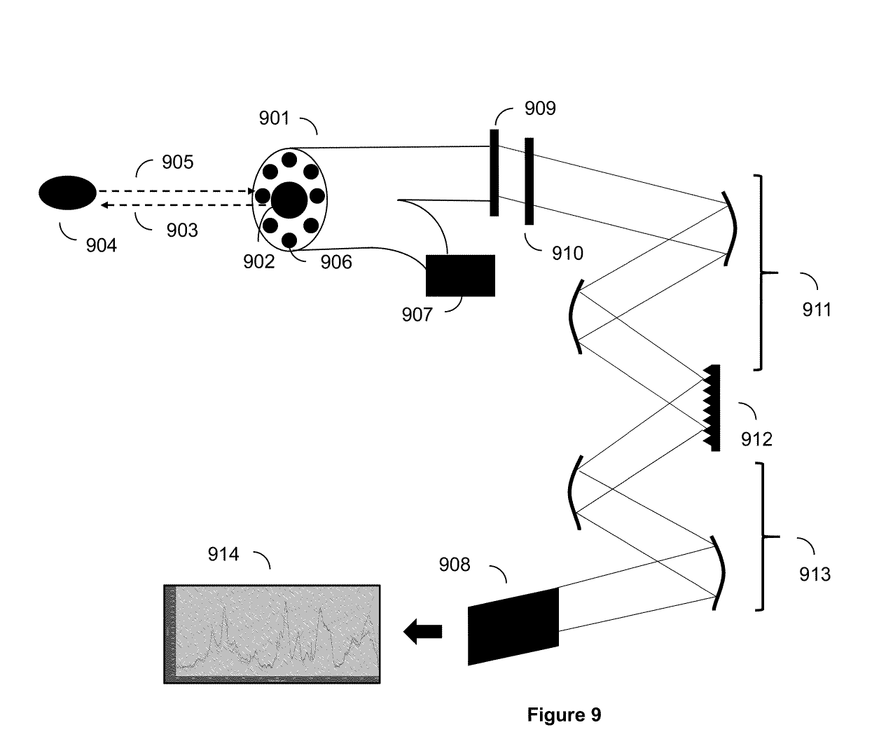

FIG. 8 and FIG. 9 provide schematics of a Raman system using transmissive grating and reflective grating, respectively, that may be used intraoperatively during neurosurgery for BTSC removal. The schematics are for illustrative purposes only and are not intended to limit the scope of the patent. The systems are largely similar with respect to the components each has. The Raman system begins with the fiber bundle 801, 901. The fiber bundle encloses the excitation fiber 802, 902 at the center. The excitation fiber directs laser 803, 903 at a certain wavelength at the target sample 804, 904, such as a cell. The laser interacts with the target sample where approximately 1 in 10⁷ photons will experience an energy change and inelastically scatter back 805, 905 in a different wavelength, or Raman shift, which is detected by the detection fibers 806, 906 on the periphery of the fiber bundle. The fiber bundle is divided into two channels, one goes to the laser box 807, 907 which generates laser at a specific wavelength. The other channel directs the refracted light towards the detector 808, 908. As the refracted light goes from the detection fiber to the detector, it goes through the slit 809, 909 which determines the resolution and a filter 810, 910 to filter out the laser line. In the transmissive grating system FIG. 8, the light then goes through lens 811 to collimate light, a transmissive grating 812 to disperse the light into different wavelengths, lens 813 to focus light, and finally to the detector 808. In the reflective grating system FIG. 9, the light goes through mirrors 911 to collimate light, a reflective grating 912 to disperse the light into different wavelengths, mirrors 913 to focus light, and finally to the detector 908. From the detector 808, 908 the refracted light is read out as a spectrum 814, 914 displaying the Raman shift that has occurred during the interaction between the laser and the target sample, producing a Raman spectral signature characteristic of the target sample 804, 904.

Returning to FIG. 3, prior to resection, the tissue is suctioned 317 and probed 318 for a Raman spectrum. The Raman spectrum of the tissue is processed 319, for example, by comparing the Raman spectrum to a database where Raman spectra have been collected from a variety of cell types including healthy brain tissues, brain tumor tissues, neural stem cells, and BTSCs. Based on the extent of the similarity of the Raman spectrum of the probed tissue to that of the existing Raman database, a probability score is generated 320 which predicts whether BTSCs are present within the suctioned tissue 321.

If BTSCs are not present within the probed tissue 321, the surgeon may relieve the suction and return the tissue 322 without harm. During the initial probing of the tissue 318 for its Raman spectrum, the tissue coordinates may also be recorded 323 indicating where the tissue was within the context of the tumor and the brain. This information is valuable if the probed tissue is resected and subjected to pathological analysis 324 where the Raman spectrum and the histological data can be compared 325 to confirm whether the resected tissue contained BTSCs. The pathology and Raman spectral data may then be added to a growing Raman database 326 which will increase in accuracy and reliability as more cases are performed.

### C. Surgery

If BTSCs are present within the suctioned tissue, the surgeon resects 327 the suctioned tissue.

Once resection is completed 315, the tissue is decannulated 328 by removing the port and any tracking instruments from the brain. Finally, the surgeon closes the dura and completes the craniotomy 329.

### D. Ex Vivo Diagnostic Imaging

The resected tissue is collected into a collection container 330. In the container, the tissue is dissociated by mechanical and enzymatic means, such as trypsin, collagenase, or accutase to generate a single cell suspension 330. The single cell suspension from the resected tissue may be probed again 331 for another confirmatory Raman measurement through a fluidic system, such as system microfluidics, tube, or capillary. The post-resection probing of resected tissue 331 may be used as a second sorting point 332 to further purify the target cell or tissue type 333, such as BTSC, from the non-targets, such as non-BTSCs 334. The collected samples 333, 334 can then be used for downstream applications such as processing in the laboratory.

### EXAMPLE 2

### Maintaining Physiological Conditions and Verification by Second Measurement

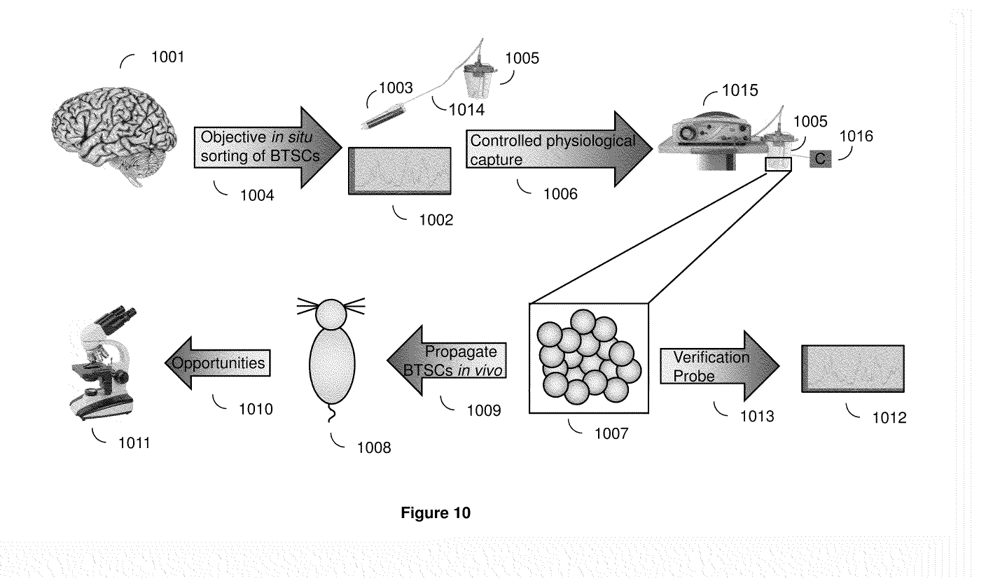

To overcome the limitations of target cell isolation described for FIG. 1, we provide an alternate workflow in FIG. 10. In the example provided herein the target cells are BTSCs, but the process can be applied to other targeted cell types as will be recognized by those skilled in the art.

The workflow begins with a subject exhibiting a brain tumor 1001. Molecular imaging using optical spectroscopy, such as Raman Spectroscopy 1002, is used as an optical spectroscopic technique to assist and guide neurosurgeons towards BTSCs.

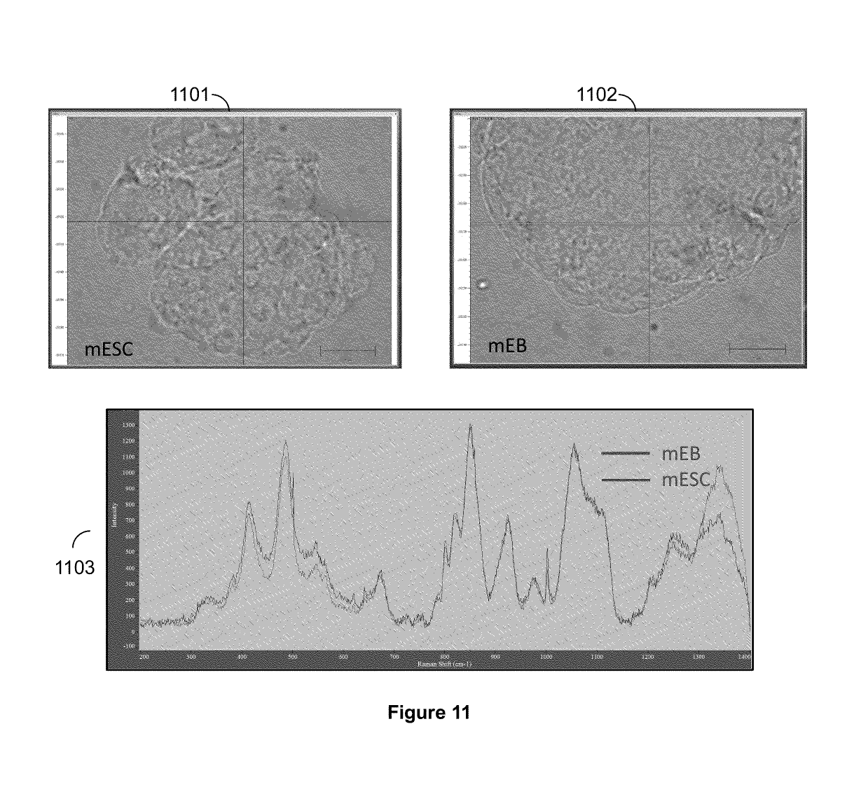

Optical spectroscopy is used to distinguish stem cells from differentiated cells types. For example FIG. 11 illustrates Raman spectra from stem cells and their derivatives. Shown in the top panels are light microscopic images of mouse embryonic stem cells (mESCs) 1101 and mouse embryoid bodies (mEB) 1102, with the latter being a differentiated cell type from the former. A crosshair in the center of microscopic images allows accurate location of Raman spectra to be acquired at cellular resolution. In the bottom panel, Raman spectra 1103 of mESCs and mEB are shown. The Raman spectra is accumulated using a confocal Raman spectrometer coupled with a microscope using an excitation wavelength of 785 nm, an accumulation time of 180 seconds done 4 times per point. In this example, 9 separate points were measured in total for each sample.

Returning to FIG. 10, the tool to probe for Raman signal is a relatively small device that can be integrated with current surgical resection tools, such as, but not limited to, NICO Myriad 1003, allowing BTSCs that have been identified by the Raman probe to be resected if desired, as described in detail in Example 1. Altogether, the integration of Raman spectroscopy 1002 with current surgical tools 1003 provides surgeons with an objective method to sort and remove BTSCs in situ 1004. Once BTSCs are captured in the resection tool, the cells are contained in such a manner that they are not exposed to the outside atmosphere such as, but not limited to, keeping the cells in a container 1005 where the atmosphere can be regulated to be similar to in vivo conditions allowing controlled physiological capture 1006. The conditions that can be regulated include, but are not limited to, growth factors (FGF2 and EGF), ECM (laminin, integrin, Fibronectin, poly-L-omithine), oxygen tension (controlled by nitrogen and oxygen levels), carbon dioxide, temperature, pH, and other factors that are crucial to maintenance of target cell in vivo characteristics, such as stem cell renewal and multipotency.

Once the BTSCs are collected in the container 1007, they can be re-probed by optical spectroscopy such as Raman spectroscopy 1012, thereby providing a verification measurement 1013.

Since the BTSCs are sorted in situ, there is no need for further isolation and expansion in vitro. For this reason, the BTSCs 1007 may be directly transplanted into xenografts 1008 where the cells can be propagated in vivo 1009 and serially transplanted as described above, thereby overcoming the limitation of extra steps of separating stem cells from tumor cells between tissue resection and cell expansion. The isolation of BTSCs from the xenografts may again be done by optical spectroscopy, such as Raman spectroscopy, for in situ sorting of BTSCs.

Therefore, returning to FIG. 1, molecular imaging using optical spectroscopy, such as Raman spectroscopy, and direct BTSC removal or sorting in situ allows the BTSCs to be directly transferred 115 into a xenograft model in a closed system and thereby overcomes the need to culture BTSCs in vitro.

Returning to FIG. 10, the BTSCs isolated as described in FIG. 10 can be used for downstream applications 1010 such as research 1011.

The proposed workflow of isolating BTSCs in FIG. 10 addresses the two limitations of the current state of the art illustrated in FIG. 1. The first limitation 113 is the inability to remove BTSCs in a targeted manner. By taking advantage of Raman spectroscopy, the workflow provides a method to sort for targeted cells in situ. The second limitation 114 is the requirement to isolate and expand BTSCs in vitro which can subject BTSCs to in vitro artifacts rendering them to not be a true representative of their in vivo counterparts. By using optical spectroscopy, such as Raman spectroscopy, to perform in situ sorting of BTSCs, it is not necessary to isolate and expand BTSCs in vitro. Furthermore, BTSCs sorted in situ can be captured in a completely closed system where environmental factors can be regulated keeping BTSCs in conditions closely resembling their in vivo environment. BTSCs isolated in this manner are a significant resource and unmet need in the market.

### EXAMPLE 3

### Isolating Target Cells from Tumor Tissue

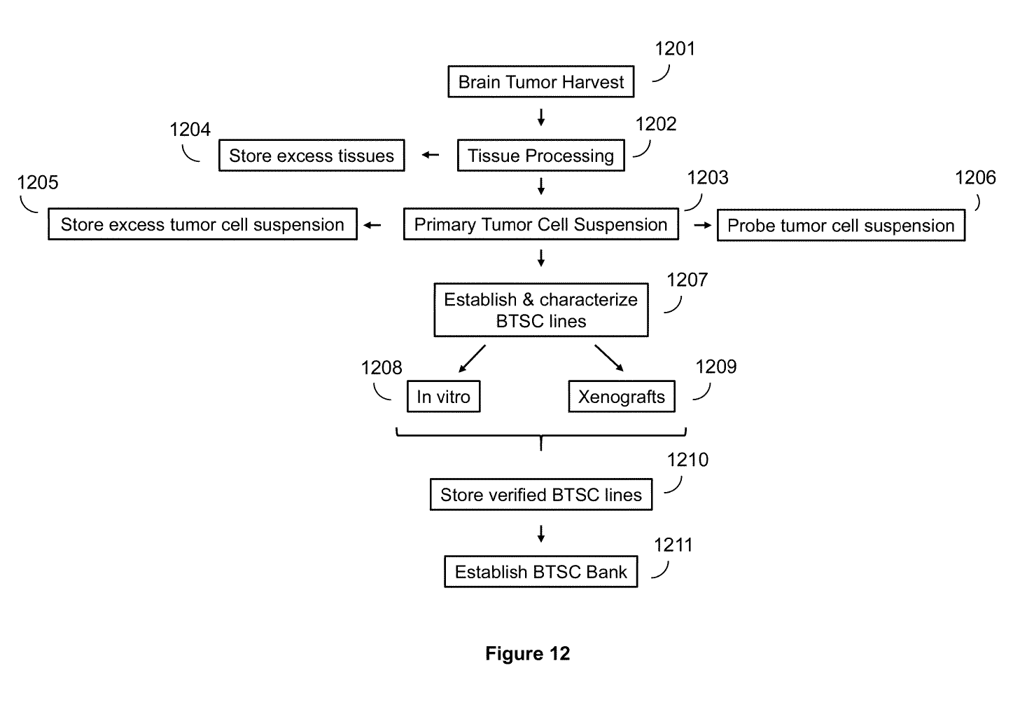

FIG. 12 illustrates a flow chart that describes how BTSCs are harvested and isolated from brain tumor samples. The flow chart begins with a brain tumor sample being harvested 1201 as described in detail above. Once the brain tumor is removed, the brain tumor sample 1201 is processed 1202 which aims to dissociate the intact tissue into a single cell suspension designated as the primary tumor cell suspension 1203. The processing 1202 of the brain tumor is done by mechanical and enzymatic dissociation. Enzymes that may be used to perform this include trypsin, collagenase, or accutase. Once a primary tumor cell suspension is made, any excess brain tumor sample may be stored 1204 for future use such as rederiving the primary tumor cell suspension. Excess primary tumor cell suspension may be stored 1205 for future uses such as rederiving BTSC lines.

The single cell suspension may be probed again 1206 for another Raman measurement through a fluidic system, such as system microfluidics, tube, or capillary (Lau et al., Lab Chip, 2008). The post-resection probing of resected tissue is used as a second sorting point to further purify the target cell or tissue type, such as BTSC, from the non-targets, such as non-BTSCs.

The BTSCs thereby isolated from the primary tumor cell suspension 1203 may then be used to establish BTSC lines 1207 as described above using either an adherent method to generate a monolayer or non-adherent method to generate neurospheres. The isolated cells may also be directly characterized in vitro 1208 and in vivo in xenografts 1209 for stem cell properties including self-renewal and multipotency. BTSCs can be stored 1210 in cold storage such as liquid nitrogen. Stored BTSC lines can also be used to establish a BTSC bank 1211 where BTSCs are catalogued and linked with subject information or other samples such as electronic medical records or subject serum, respectively.

Thus, BTSCs can be isolated from brain tumor using optical spectroscopy to identify BTSCs in situ and to subsequently isolate BTSCs from other tumor cells, thereby eliminating the need to culture the primary tumor cell suspension in order to purify BTSCs. The method outlined in FIG. 12 can be applied to the isolation of other target cells, such as other CSCs or adult stem cells.

1. A method for isolating at least one target cell from at least one cell in a tissue of a subject, the method comprising:
accessing the tissue using an access corridor;
measuring spectra of the at least one cell in the tissue using optical spectroscopy via the access corridor, thereby providing measured spectra of the at least one cell;
comparing the measured spectra of the at least one cell in the tissue to at least one of a database of signature spectra and spectra of cells in surrounding tissue, thereby providing spectral information of the at least one cell;
calculating a probability score to predict whether the at least one target cell is present in the tissue;
using the spectral information of the at least one cell and the probability score to identify the at least one target cell in the tissue, thereby providing at least one identified target cell; and
resecting the at least one identified target cell into a container.

2. A method for isolating and verifying at least one target cell from at least one cell in a tissue of a subject, the method comprising:
accessing the tissue using an access corridor;
measuring spectra of the at least one cell in the tissue using optical spectroscopy via the access corridor, thereby providing measured spectra of the at least one cell;
comparing the measured spectra of the at least one cell in the tissue to at least one of a database of signature spectra and spectra of cells in surrounding tissue, thereby providing spectral information of the at least one cell;
calculating a probability score to predict whether the at least one target cell is present in the tissue;
using the spectral information of the at least one cell and the probability score to identify the at least one target cell in the tissue, thereby providing at least one identified target cell;
resecting the at least one cell comprising the at least one identified target cell, thereby providing at least one resected cell;
collecting the at least one resected cell through a collection tube into a collection container; and
measuring spectra of the at least one resected cell in at least one of the collection tube and the collection container, thereby providing measured spectra of the at least one resected cell.

3. The method of claim 2, further comprising using the measured spectra of the at least one resected cell in at least one of the collection tube and the collection container to sort the at least one identified target cell from the at least one resected cell.

4. The method of claim 3, further comprising assaying the at least one identified target cell in a model comprising at least one of a cell culture and animal model.

5. The method of claim 1, wherein using the spectral information of the at least one cell and the probability score to identify the at least one target cell in the tissue comprises identifying at least one of a cancer cell, a stem cell, and a neural cell.

6. The method of claim 1, wherein comparing further comprises using the measured spectra of the at least one cell to identify at least one target cell, using the measured spectra of the at least one cell comprising at least one of:
determining whether the measured spectra of the at least one cell is within a predetermined range of the signature spectra; and
determining whether the measured spectra of the at least one cell is different than the spectra of the cells in the surrounding tissue.

7. The method of claim 2, wherein comparing further comprises using the measured spectra of the at least one cell to identify at least one target cell, using the measured spectra of the at least one cell comprising at least one of:
determining whether the measured spectra of the at least one cell is within a predetermined range of the signature spectra; and
determining whether the measured spectra of the at least one cell is different than the spectra of the cells in the surrounding tissue.

8. The method of claim 1, wherein measuring the spectra of the at least one cell in the tissue, using optical spectroscopy via the access corridor, comprises using a sterile probe, using the sterile probe comprising using a probe covered with a sterile waterproof disposable sleeve.

9. The method of claim 1, wherein using the optical spectroscopy comprises using Raman spectroscopy.

10. The method of claim 1, wherein using the access corridor comprises using a surgical port.

11. The method of claim 1, wherein each of measuring the spectra of the at least one cell, comparing the measured spectra, calculating a probability score, and using the spectral information to identify the at least one target cell in the tissue comprises intraoperatively performing each such step.

12. The method of claim 1, wherein each of measuring the spectra of the at least one cell, comparing the measured spectra, calculating a probability score, and using the spectral information to identify the at least one target cell in the tissue comprises intraoperatively performing each such step and recording a set of positional coordinates for the at least one target cell in the tissue.

13. The method of claim 2, wherein using the spectral information of the at least one cell and the probability score to identify the at least one target cell in the tissue comprises identifying at least one of a cancer cell, a stem cell, and a neural cell.

14. The method of claim 2, wherein measuring the spectra of the at least one cell in the tissue comprises using a sterile probe, using the sterile probe comprising using a probe covered with a sterile waterproof disposable sleeve.

15. The method of claim 2, wherein using the optical spectroscopy comprises using Raman spectroscopy.

16. The method of claim 2, wherein using the access corridor comprises using a surgical port.

17. The method of claim 2, wherein each of measuring the spectra of the at least one cell, comparing the measured spectra, calculating a probability score, and using the spectral information to identify the at least one target cell in the tissue comprises intraoperatively performing each such step.

18. The method of claim 2, wherein each of measuring the spectra of the at least one cell, comparing the measured spectra, calculating a probability score, and using the spectral information to identify the at least one target cell in the tissue comprises intraoperatively performing each such step and recording a set of positional coordinates for the at least one target cell in the tissue.

19. A system for identifying at least one target cell from at least one cell in a tissue of a subject, comprising:
an access corridor for accessing the at least one cell;
a probe connected to means for measuring spectra of the at least one cell by optical spectroscopy through the access corridor, the measuring means providing measured spectra of the at least one cell;
a processor configured to compare the measured spectra of the at least one cell and to calculate a probability score;
means for maintaining the probe in a sterile condition, the maintaining means comprising at least one of a sterilizable probe outer surface and a sterilizable probe outer sleeve;
a resection tool for providing suction and resection of the tissue through the access corridor, whereby resected tissue is providable;
a collection container for holding the resected tissue; and
a collection tube configured to couple the resection tool with the collection container.

20. The system of claim 19, wherein the access corridor comprises a surgical port.

21. The system of claim 19, wherein the measuring means comprises a Raman spectroscope.

22. The system of claim 19, wherein the maintaining means comprises a sterile waterproof disposable sleeve.

23. The system of claim 19, wherein at least one of the collection tube and the collection container comprises means for performing a second optical spectroscopic measurement.

24. The system of claim 19, wherein the collection container is configured to regulate at least one of: at least one growth factor, an oxygen tension by way of controlling a nitrogen level and an oxygen levels, a carbon dioxide level, a temperature, a pH, and any other parameter crucial to in vivo maintenance of the at least one target cell for maintaining the resected tissue in a physiologic condition.

25. (canceled)

26. The method of claim 1, wherein the collection container is configured to maintain the at least one resected cell in a physiologic condition.

27. The method of claim 2, wherein the collection container is configured to maintain the at least one resected cell in a physiologic condition.
